(19) United States
(12) Patent Application Publication
(10) Pub. No.: US 2010/0022568 A1
(43) Pub. Date: Jan. 28, 2010

(54) ENDOTHELIN RECEPTOR ANTAGONISTS FOR EARLY STAGE IDIOPATHIC PULMONARY FIBROSIS

(75) Inventors: Martine Clozel, Binningen (CH); John Gatfield, Basel (CH); Sebastien Roux, Basel (CH)

Correspondence Address: HOXIE & ASSOCIATES LLC, 75 MAIN STREET , SUITE 301, MILLBURN, NJ, 07041, US

(73) Assignee: Actelion Pharmaceeuticals Ltd., Allschwil (CH)

(21) Appl. No.: 12/296,895

(22) PCT Filed: Apr. 12, 2007
(86) PCT No.: PCT/IB07/51328

§ 371 Date: Oct. 10, 2008

(30) Foreign Application Priority Data
Apr. 13, 2006 (IB) PCT/IB2006/051170
May 19, 2006 (IB) PCT/IB2006/051610

(51) Int. Cl. A61K 31/506; A61K 31/505; A61K 31/42; A61P 11/00

(52) U.S. Cl. 514/274; 514/269; 514/378

(57) ABSTRACT

This present invention relates to the use of an endothelin receptor antagonist for the preparation of a medicament for the treatment of early stage idiopathic pulmonary fibrosis.

The present invention relates to the use of endothelin receptor antagonists (hereinafter ERA) for the treatment of early stage idiopathic pulmonary fibrosis (hereinafter early stage IPF or early IPF).

Idiopathic pulmonary fibrosis (IPF), also known as cryptogenic fibrosing alveolitis, is a distinct clinical disorder belonging to the spectrum of interstitial lung diseases (ILD). IPF is a progressive disease characterized by the presence of a histological pattern of usual interstitial pneumonia (UIP) on surgical lung biopsy. IPF was used to be considered as a chronic inflammatory disease resulting in parenchymal fibrosis. However, recent evidence suggests a mechanism of abnormal wound healing, with progressive extracellular matrix accumulation, decreased fibroblast-myoblast cell death, continuous epithelial cell apoptosis and abnormal re-epithelialization. Progressive fibrotic tissue deposition in the interstitial areas of the lung leads to decreased lung compliance and reduced gas exchanges.

The onset of symptoms is usually gradual and patients complain of non-productive cough, shortness of breath occurring first on exercise and then at rest. Cyanosis, cor pulmonale, and peripheral edema may be observed in the late phase of the disease.

In the presence of a surgical lung biopsy showing the histological appearance of UIP, the definite diagnosis of IPF requires the following (American Thoracic Society. Idiopathic pulmonary fibrosis: diagnosis and treatment. International consensus statement. American Thoracic Society (ATS) and the European Respiratory Society (ERS). Am J Respir Crit Care Med 2000; 161:646-64):
1) The exclusion of other causes of ILD,
2) Abnormal pulmonary function studies that include evidence of restriction of lung capacity and/or impaired gas exchange or decreased diffusing capacity for carbon monoxide (DLCO),
3) Abnormalities on conventional chest radiograph or high-resolution computed tomography (HRCT) scans.

The criteria for diagnosis of IPF in the absence of a surgical lung biopsy necessitate the correlation between all clinical and radiological features.

According to LeadDiscovery (2006), Idiopathic pulmonary fibrosis (hereinafter IPF) is a devastating, relentlessly progressive and lethal disease for which current therapy is minimally effective.

Precise figures for prevalence and incidence of IPF have not been reported. Prevalence was thought to be between 3 and 6 cases per 100,000 but could be as high as 13 to 20 cases per 100,000. Prevalence is higher in older adults (two-thirds of patients are over 60 years of age) and in males. The median survival after the diagnosis of biopsy-confirmed IPF is less than 3 years.

No therapies have been shown to improve survival or quality of life for patients with IPF. Current treatment is still based on the former presumption that IPF is an inflammatory process with concurrent remodeling of the lung by fibrosis. Consequently, it involves anti-inflammatory therapy, including corticosteroids, immunosuppressive/cytotoxic agents (e.g. azathioprine, cyclophosphamide) or a combination of both. However, because of the marginal benefit and serious side effects of the current therapies, along with newer insights into the pathogenesis of IPF, novel therapeutic approaches are highly needed. Antifibrotic therapy is aimed at decreasing matrix deposition or increasing collagen breakdown and a number of agents including colchicine, D-penicillamine, interferon gamma, and pirfenidone are currently under investigation. Lung transplantation has emerged as a viable option for some patients with IPF.

The neurohormone endothelin-1 (ET-1) belongs to a family of 21-amino-acid peptides released from the endothelium and is one of the most potent vasoconstrictors known. ET-1 can also promote fibrosis, cell proliferation, and remodeling, and is pro-inflammatory. ET-1 can modulate matrix production and turnover by altering the metabolism of fibroblasts to stimulate collagen synthesis or decrease interstitial collagenase production. Activation of the paracrine lung ET system has been confirmed in animal models of pulmonary fibrosis. ET-1 has also been linked to IPF in humans. In patients with IPF, ET-1 is increased in airway epithelium, and type TI pneumocytes, compared with control subjects and with patients with nonspecific fibrosis. Thus ET-1 could be a major player in the pathogenesis of IPF.

High Resolution Computer Tomography (HRCT) as well as classical computer tomography (CT) are to date together with pulmonary function tests the best non invasive tools to assess the extent of the disease and to attempt to delineate its stage of progression. Typically IPF at start of the disease will mainly show on CT scan ground-glass attenuation with little or no honeycomb. Ground-glass attenuation corresponds histologically to patchy alveolar septal fibrosis, air space filling with macrophages with interstitial inflammation. At a later stage ground-glass will be substituted by more reticular opacities and honeycomb. The latter corresponds to the destruction of the lung with dilatation of bronchioles that communicate with proximal airways. Honeycomb lesions tend to enlarge slowly over time (King Jr. T E. Idiopathic interstitial pneumonias in Interstitial Lung Disease fourth edition pages 701 786 Schwartz, King editors 2003 BC Decker Inc Hamilton-London).

Honeycomb can be semi-quantitated on HRCT at the lobe level or zones with scales from 0 to 5 or 0 to 100 with increments of 5 (Lynch D A et al. Am J Respir Crit Care Med 2005 172 488-493; Akira M, et al Idiopathic pulmonary fibrosis: progression of honeycombing at thin-section CT Radiology 1993 189: 687-691).

Early stage of IPF can be at best characterized by the presence of no or little honeycomb on HRCT or CT scans, as well as the presence of ground-glass in one or both lungs but not limited to these features. Early stage of IPF can be more accurately defined as IPF associated with no or low honeycomb at time of disease diagnosis. In rare cases the HRCT will not show ground-glass attenuation and/or honeycomb and/or reticulation. However, early IPF may also be diagnosed by other usual diagnostic tools but not limited to, such as magnetic resonance imaging, broncho-alveolar lavage, lung biopsy for histological assessment (e.g. surgical, transbronchial, or via mediastinoscopy).

Additionally, early IPF may also be diagnosed by cardio-pulmonary exercise test.

Despite low or no honeycomb visible on HRCT scan, honeycomb still may be seen on histological sections.

The term “low honeycomb” or “little honeycomb” means that honeycomb is present in less than 25% of the overall lung fields. In a further embodiment, the term “low honeycomb” or “little honeycomb” means that honeycomb is present in less than 10% of the overall lung fields.

According to LeadDiscovery (2006), diagnosing patients with early-stage IPF remains a great challenge.

Bosentan (Tracleer®) is an oral treatment for PAH (Class III and IV in the United States, Class III in Europe). Bosentan is a dual endothelin receptor antagonist with affinity for both endothelin ET_{A} and ET_{B} receptors thereby preventing the deleterious effects of ET-1. Bosentan competes with the binding of ET-1 to both ETA and ETB receptors with a slightly higher affinity for ET_{A} receptors (Ki=4.1-43 nM) than for ET_{B} receptors (Ki=38-730 nM).

In a clinical study (BUILD-1), the efficacy of bosentan in patients suffering from idiopathic pulmonary fibrosis (IPF) was evaluated in 2003. The studies did not show an effect on the primary endpoint of exercise capacity. However, bosentan showed efficacy on secondary endpoints related to death or disease worsening, providing strong rationale for Phase III mortality/morbidity study in IPF.

Full analysis of the BUILD-1 study presented at the American Thoracic Society (ATS) conference (23.05.2006) included evaluating the treatment effect of bosentan in patients who had lung biopsy (n=99) as a proof of IPF. The BUILD-1 findings in lung-biopsy proven IPF are unexpected, and warrant further clinical evaluation of bosentan in this indication. A phase III mortality and morbidity study in patients with biopsy proven IPF (BUILD-3 study) started by the end of 2006 and is currently ongoing.

WO 2004/105684 describes the use of a combination of NAC, SAPK and bosentan for IPF. However, early stage IPF is not mentioned in the publication.

WO 2005/110478 describes the use of a combination of pirfenidone or a pirfenidone analog and bosentan for IPF. Additionally, WO 2005/110478 describes the use of a combination of IFN-gamma and bosentan for IPF. However, early stage IPF is not mentioned in the publication.

Surprisingly, we found that this efficacy of bosentan was restricted to patients with early stage IPF. Thus, bosentan is useful for the treatment of early stage IPF. Further tests that have been carried out demonstrate that other ERA's are also useful for the treatment of early stage IPF.

The present invention relates to the use of an endothelin receptor antagonist, or a pharmaceutical composition comprising an endothelin receptor antagonist and either pirfenidone or interferon-gamma, for the preparation of a medicament for the treatment of early stage idiopathic pulmonary fibrosis.

A further embodiment of the present invention relates to the above-described use wherein the endothelin receptor antagonist is a dual endothelin receptor antagonist or a mixed endothelin receptor antagonist.

A further embodiment of the present invention relates to the above-described use wherein the endothelin receptor antagonist is a selective endothelin receptor antagonist that binds selectively to the ET_{A} receptor.

A further embodiment of the present invention relates to the above-described use wherein the endothelin receptor antagonist is a selective endothelin receptor antagonist that binds selectively to the ET_{B} receptor.

A further embodiment of the present invention relates to the above-described use wherein the endothelin receptor antagonist is selected from table 1.

A further embodiment of the present invention relates to the above-described use wherein the endothelin receptor antagonist is selected from darusentan, ambrisentan, atrasentan, sitaxsentan, avosentan, TBC-3711, tezosentan, clazosentan, propyl-sulfamic acid {5-(4-bromo-phenyl)-6-[2-(5-bromo-pyrimidin-2-yloxy)-ethoxy]-pyrimidine-4-yl}-amide and bosentan.

A further embodiment of the present invention relates to the above-described use wherein the endothelin receptor antagonist is selected from darusentan, ambrisentan, sitaxsentan, avosentan, TBC-3711, propyl-sulfamic acid {5-(4-bromo-phenyl)-6-[2-(5-bromo-pyrimidin-2-yloxy)-ethoxy]-pyrimidine-4-yl}-amide and bosentan.

A further embodiment of the present invention relates to the above-described use wherein the endothelin receptor antagonist is bosentan.

A further embodiment of the present invention relates to the above-described use wherein honeycomb on HRCT or CT scans is either absent or minimal.

A further embodiment of the present invention relates to the above-described use wherein honeycomb on HRCT or CT scans is present in less than 25% of the overall lung fields.

A further embodiment of the present invention relates to the above-described use wherein honeycomb on HRCT or CT scans is present in less than 10% of the overall lung fields.

A further embodiment of the present invention relates to the above-described use wherein the ground-glass attenuation could be any percentage between above zero to 80% of lung fields.

A further embodiment of the present invention relates to the above-described use wherein bosentan is given to a patient at a daily dosage of 125 mg with or without a lower starting dose.

A further embodiment of the present invention relates to the above-described use wherein bosentan is given to a patient at a daily dosage of 250 mg with or without a lower starting dose.

The present invention relates to the use of an endothelin receptor antagonist alone or in combination with interferon-gamma (e.g. interferon gamma-1b) or pirfenidone for the preparation of a medicament for the treatment of early stage IPF.

Pirfenidone and interferon-gamma (e.g. interferon gamma-1b) can be purchased from commercial suppliers or synthesized according to methods in the art.

Early stage of IPF can be delineated as a stage of the disease at which honeycomb on HRCT or CT scans is either absent or minimal. In an embodiment of the invention the honeycomb is present in less than 10% of the overall lung fields. In a preferred embodiment the honeycomb, when expressed in a 0 to 100% scale, is present in less than 8%, or less than 5%, or less than 3%, or less than 2% of the overall lung fields. Most preferred the honeycomb is present in less than 1% of the overall lung fields. In a further embodiment the honeycomb, when expressed in a 1 to 5 scale, is present in less than a score of 3, preferably less than a score of 2, most preferred less than a score of 1.

An additional feature is the presence of ground-glass attenuation in one or both lungs fields but not limited to these features. Ground-glass extent in early IPF could be any percentage between above zero to 80%, preferably more than 2% to up to 80% of lung fields (Akira M, et al Idiopathic pulmonary fibrosis: progression of honeycombing at thin-section CT Radiology 1993 189: 687-691).

When IPF cannot yet with high certainty be diagnosed by clinical/radiological features expressed in the ATS/ERS consensus guidelines, typically a lung biopsy is performed to either rule out or confirm early stage IPF (reference: American Thoracic Society. Idiopathic pulmonary fibrosis: diagnosis and treatment. International consensus statement. American Thoracic Society (ATS) and the European Respiratory Society (ERS). Am J Respir Crit Care Med 2000; 161:646-64).

Endothelin Receptor Antagonists (ERA):

Endothelin receptor antagonists, as defined above, encompass a wide range of structures and are useful alone or in the combinations and methods of the present invention. Nonlimiting examples of endothelin receptor antagonists that may be used in the present invention include those endothelin receptor antagonists as disclosed below. The endothelin receptor antagonist references identified below are incorporated herein in their entirety.

Endothelin-1 is a potent endogenous vasoconstrictor and smooth-muscle mitogen that is overexpressed in the plasma and lung tissue of patients with pulmonary arterial hypertension and pulmonary fibrosis. There are two classes of endothelin receptors: ET_{A} receptors and ET_{B} receptors, which play significantly different roles in regulating blood vessel diameter. In chronic pathological situations, the pathological effects of ET-1 can be mediated via both ET_{A} and ET_{B} receptors.

Two types of ERAs have been developed: dual ERAs, which block both ET_{A} and ET_{B} receptors, and selective ERAs, which block only ET_{A} receptors.

Dual Endothelin Receptor Antagonist (also called mixed Endothelin Receptor Antagonist) block both the ET_{A} and ET_{B} receptors. Bosentan (Tracleer®) is the first FDA approved ERA (see U.S. Pat. No. 5,292, 740 or U.S. Pat. No. 5,883,254; incorporated herein in its entirety by reference thereto).

Selective ERAs bind to the ET_{A} receptor in preference to the ET_{B} receptor. Currently, there are selective ERAs in clinical trials, such as sitaxsentan, atrasentan, avosentan, ambrisentan (BSF 208075), and TBC3711.

The synthesis of Ambrisentan is described in U.S. Pat. No. 5,932,730 and U.S. Pat. No. 5,969,134.

The synthesis of propyl-sulfamic acid {5-(4-bromo-phenyl)-6-[2-(5-bromo-pyrimidin-2-yloxy)-ethoxy]-pyrimidine-4-yl}-amide is described in WO 2002/53557.

TABLE 1
Endothelin Receptor Antagonists

| COMPOUNDS AND COMPOUND | |
|---|---|
| CLASSES | REFERENCE/MANUFACTURER |
| bosentan | U.S. Pat. No. 5,883,254; (CAS No. |
| | 157212-55-0); Roche Holding |
| | AG, Actelion, Genentech |
| sitaxsentan | U.S. Pat. No. 5,594,021; (CAS No. |
| | 184036-34-8); ICOS-Texas |
| | Biotechnology, L.P. |
| darusentan | WO 99/16446; (CAS No. 221176- |
| BMS-187308 | Bristol-Meyers Squibb; Clin. |
| | Cardiol. Vol. 23, Oct. |
| | 2000. |
| BMS-193884 | Bristol-Meyers Squibb; |
| | Pharmacotherapy 22(1): 54-65, |
| | 2002. |
| BMS-20794 | Bristol-Meyers Squibb; |
| | Pharmacotherapy 22(1): 54-65, |
| | 2002. |
| BSF-208075; ambrisentan | Abbott Laboratories, Myogen, |
| | Inc. |
| CGS-27830 | Novartis; Pharmacotherapy |
| | 22(1): 54-65, 2002. |
| IRL-3630 | Novartis; Pharmacotherapy |
| | 22(1): 54-65, 2002. |
| IRL-1038 | SmithKline Beecham |
| enrasentan | |
| FR-139317 | Fujisawa Pharmaceutical Co, |
| | Ltd.; Pharmacotherapy |
| | 22(1): 54-65, 2002. |
| J-104121 | Merck/Banyu; Pharmacotherapy |
| | 22(1): 54-65, 2002. |
| J-104132 | Merck/Banyu; Pharmacotherapy |
| | 22(1): 54-65, 2002. |
| EMD-94246 | Merck; Pharmacotherapy |
| | 22(1): 54-65, 2002. |
| L-744453 | Merck; Pharmacotherapy |
| | 22(1): 54-65, 2002. |
| L-749329 | Merck; Pharmacotherapy |
| | 22(1): 54-65, 2002. |
| L-753037 | Merck; Pharmacotherapy |
| | 22(1): 54-65, 2002. |
| L-754142 | Merck; Pharmacotherapy |
| | 22(1): 54-65, 2002. |
| LU135252 | Knoll AG; Pharmacotherapy |
| | 22(1): 54-65, 2002. |
| LU208075 | Knoll AG; Pharmacotherapy |
| | 22(1): 54-65, 2002. |
| LU302146 | Knoll AG; Pharmacotherapy |
| | 22(1): 54-65, 2002. |
| LU224332 | Knoll AG; Pharmacotherapy |
| | 22(1): 54-65, 2002. |
| LU302872 | Knoll AG; Pharmacotherapy |
| | 22(1): 54-65, 2002. |
| PD-142893 | Parke-Davis; Pharmacotherapy |
| | 22(1): 54-65, 2002. |
| PD-145065 | Parke-Davis; Pharmacotherapy |
| | 22(1): 54-65, 2002. |
| PD-147953 | Parke-Davis; Pharmacotherapy |
| | 22(1): 54-65, 2002. |
| PD-156123 | WO95/05376 |
| RO46-2005 | Hoffmann-La Roche; |
| | Pharmacotherapy 22(1): 54-65, |
| | 2002. |
| RO47-0203 | Hoffmann-La Roche; |
| | Pharmacotherapy 22(1): 54-65, |
| | 2002. |
| RO 48-5695 | Hoffmann-La Roche; |
| | Pharmacotherapy 22(1): 54-65, |
| | 2002. |
| RO 61-1790 | Hoffmann-La Roche; |
| | Pharmacotherapy 22(1): 54-65, |
| | 2002. |
| RO-61-0612 | Roche; Clin. Cardiol. Vol. |
| | 23, Oct. 2000. |
| SB-209670 | SmithKline Beecham; |
| | Pharmacotherapy 22(1): 54-65, |
| | 2002. |
| SB-217242 | SmithKline Beecham; |
| | Pharmacotherapy 22(1): 54-65, |
| | 2002. |
| SB-234551 | SmithKline Beecham; |
| | Pharmacotherapy 22(1): 54-65, |
| | 2002. |
| SB-247083 | SmithKline Beecham; |
| | Pharmacotherapy 22(1): 54-65, |
| | 2002. |
| TA-0115 | Tanabe Seiyaku Co.; |
| | Pharmacotherapy 22(1): 54-65, |
| | 2002. |
| TA-0201 | Tanabe Seiyaku Co.; |
| | Pharmacotherapy 22(1): 54-65, |
| | 2002. |
| TBC11251 | Texas Biotechnology Co.; |
| | Pharmacotherapy 22(1): 54-65, |
| | 2002. |
| TBC-3711 | Texas Biotechnology Co. |
| TBC-11251 | Texas Biotechnology Co.; Clin. |
| | Cardio. Vol. 23, Oct. 2000. |
| ZD 1611 | Zeneca Group plc.; |
| | Pharmacotherapy 22(1): 54-65, |
| | 2002. |
| Sulphisoxazole (4-Amino-N- | (CAS No. 127-69-5); Biochem. |
| (3,4-dimethyl-5-isoxazolyl) | Biophys. Res. Comm. 201 228 |
| benzenesulfonamide) | |
| Sulfonamide derivatives | WO 01/049685; Texas |
| | Biotechnology Corp. |
| 3-Sulfamoyl-pyrazole | EP 1072597; Pfizer Ltd. |
| derivatives | |
| Biphenyl isoxazole | U.S. Pat. No. 6,313,308, WO 00/056685; |
| sulfonamide compounds | Bristol Myers Squibb Co. |
| 4-Heterocyclyl-sulfonamidyl- | WO 00/052007; Hoffmann LaRoche |
| 6-methoxy-5-(2- | & Co. |
| methoxyphenoxy)-2-pyridyl- | |
| pyrimidine derivatives and | |
| their salts | |
| 3-acylamino-propionic acid | EP 1140867; BASF AG |
| and 3-sulfonylamino-propionic | |
| acid derivatives | |
| Phenylsulfonamide derivatives | U.S. Pat. No. 6,107,320; Bristol-Myers |
| and their salts | Squibb Co. |
| Pyrrole derivatives and their | JP 2000063354; Sumitomo |
| acid and alkali salts | Seiyaku, KK |
| Furanone and thiophenone | U.S. Pat. No. 6,017,916; Warner Lambert |
| derivatives | Co. |
| Pyrimidyl sulfonamide | EP 959072; Tanabe Seiyaku Co. |
| derivatives | |
| Pyrimidyl sulfonamide | EP 959073; Tanabe Seiyaku Co. |
| derivatives | |
| Benzothiazine derivatives, | GB 2337048; Warner Lambert Co. |
| their acid addition and base | |
| salts | |
| Phenyl isoxazole sulfonamide | U.S. Pat. No. 5,939,446; Bristol-Myers |
| derivatives, their | Squibb Co. |
| enantiomers, diastereomers | |
| and salts | |
| 5-benzodioxolyl- | EP 1049691, Banyu Pharm Co. |
| cyclopentenopyridine | Ltd. |
| derivatives, including 5- | |
| (2,2-Difluoro-1,3- | |
| benzodioxol-5-yl) | |
| cyclopentenopyridine | |
| derivatives and (5S,6R,7R)- | |
| 6-carboxy-5-(2,2-difluoro- | |
| 1,3-benzodioxol-5-yl)-7-(2- | |
| (3-hydroxy-2-methylpropyl)-4- | |
| methoxyphenyl)-2-N- | |
| isopropylaminocyclopentene (1, | |
| 2-b)pyridine | |
| Amino acid derivatives and | U.S. Pat. No. 5,922,681; Warner Lambert |
| their salts including (R-(R*, | Co. |
| S*))-gamma-((3-(1H-indol-3- | |
| yl)-2-methyl-1-oxo-2- | |
| (((tricyclo(3.3.1.13,7)dec-2- | |
| yloxy)carbonyl)amino) | |
| propyl)amino)- | |
| benzenepentanoic acid | |
| 15-ketoprostaglandin E | U.S. Pat. No. 6,197,821, EP 978284; R- |
| compound provided that it | Tech Ueno Ltd. |
| does not contain an alpha | |
| bonded 8C or more backbone, | |
| including 13,14-dihydro-15- | |
| keto-16,16-difluoro-18S- | |
| methylprostaglandin E1 | |
| Pyridyl-thiazole derivatives | U.S. Pat. No. 5,891,892; Warner Lambert |
| | Co. |
| Pyrrolidine and piperidine | U.S. Pat. No. 6,162,927, EP 1003740; |
| derivatives, their analogues | Abbott Laboratories |
| and salts | |
| Pyrrolidine carboxylic acid | U.S. Pat. No. 6,124,341, EP 991620; |
| derivatives, their salts and | Abbott Laboratories |
| stereoisomers | |
| Biphenyl derivatives of | U.S. Pat. No. 5,846,985; Bristol-Myers |
| formula (I), their | Squibb Co. |
| enantiomers, diastereomers, | |
| and salts | |
| Compound S-19777 of formula | JP 10306087; Sankyo Co. Ltd. |
| (I) | |
| Sulphonamide derivatives of | JP 10194972; Tanabe Seiyaku |
| formula (I) and their salts | Co. |
| Prostanoic acid derivative | U.S. Pat. No. 6,242,485, EP 857718; R- |
| with an alpha-chain of at | Tech Ueno Ltd. |
| least 8 skeletal C | |
| Aminoalkoxy or sulpho-alkoxy | U.S. Pat. No. 6,133,263, WO 9737986; |
| furan-2-ones or thiophen-2- | Warner Lambert Co. |
| ones, all of formula (I), and | |
| their salts | |
| Aminoalkoxy 5-hydroxyfuran-2- | U.S. Pat. No. 6,297,274, WO 9737985, |
| ones, their aminoalkylamino | Warner Lambert Co. |
| and alkyl-sulphonic acid | |
| analogues, all of formula | |
| (I), their tautomeric open- | |
| chain keto-acid forms, and | |
| their salts | |
| Pyrrolidine derivatives | EP 888340; Abbott Laboratories |
| Phenylalanine derivatives of | U.S. Pat. No. 5,658,943; Warner Lambert |
| formula (I) | Co. |
| N-isoxazolyl- | U.S. Pat. No. 6,271,248, U.S. Pat. No. 6,080,774, EP |
| biphenylsulphonamide | 768305; Bristol-Myers Squibb |
| derivatives of formula (I) | Co. |
| and their salts, including N- | |
| (3,4-di methyl-5-isoxazolyl)- | |
| 2′-(hydroxymethyl) (1,1′-bi | |
| phenyl)-2-sulphonamide | |
| 3-Aryl (or cycloalkyl) 5H- | U.S. Pat. No. 5,998,468, WO 9708169; |
| furan-2-ones of formula (I) | Warner Lambert Co. |
| and their salts, solvates, | |
| and hydrates | |
| N-Isoxazolyl-4′- | U.S. Pat. No. 5,612,359; Bristol-Myers |
| heterocyclyl(alkyl)-1,1′- | Squibb Co. |
| biphenyl-2-sulphonamides of | |
| formula (I) and their | |
| enantiomers, diastereomers | |
| and salts | |
| Thieno(2,3-d) pyrimidine | U.S. Pat. No. 6,140,325, EP 846119; |
| derivatives (I) contg. a | Takeda Chem. Ind. Ltd. |
| carboxyl gp. or ester and a | |
| gp. other than carboxyl | |
| which is capable of forming | |
| an anion or a gp. | |
| convertible to it | |
| 2(5H)-Furanone derivatives of | U.S. Pat. No. 5,922,759, U.S. Pat. No. 6,017,951, WO |
| formula (I) and their salts | 9702265; Warner Lambert Co. |
| Heterocyclic pyridine | U.S. Pat. No. 6,258,817, U.S. Pat. No. 6,060,475, U.S. Pat. No. |
| sulphonamide derivatives of | 5866568, EP 832082; ZENECA |
| formula (I) and their N | LTD. |
| oxides, salts and prodrugs | |
| Dihydropyridine carboxylic | U.S. Pat. No. 5,576,439; Ciba Geigy Corp. |
| acid anhydride derivatives of | |
| formula (I) and their salts | |
| N-pyrimidinyl-sulphonamide | U.S. Pat. No. 5,739,333, EP 743307; |
| derivatives of formula (I) | Tanabe Seiyaku Co. |
| and their salts | |
| Aroylamidoacyl di-C-substd. | U.S. Pat. No. 5,977,075, EP 821670, |
| glycine derivatives of | Novartis AG |
| formula (I) and their salts | |
| Benzothiazine dioxides of | U.S. Pat. No. 5,599,811, EP 811001; |
| formula (I) and their salts | Warner Lambert Co. |
| N-Isoxazolyl-4′-substd.-1,1′- | U.S. Pat. No. 5,760,038, EP 725067; |
| biphenyl-2-sulphonamide | Bristol-Myers Squibb Co. |
| derivatives of formula (I) | |
| and their enantiomers, | |
| diastereomers and salts | |
| 4-Oxo-2-butenoic acid | WO 9623773, JP 8523414; Banyu |
| derivatives of formula (I) | Pharm Co. Ltd. |
| and 3-hydroxy-2(5H)-furanone | |
| derivatives of formula (II), | |
| and their salts | |
| Aza-aminoacids of formula (I) | ZA 9501743; Abbott |
| | Laboratories |
| Sulphonamides of formula (I) | U.S. Pat. No. 6,004,965, EP 799209; |
| and their salts | Hoffmann La Roche & Co. |
| Aryl compounds of formula | U.S. Pat. No. 6,207,686, EP 792265; |
| (I) and their salts | Fujisawa Pharm Co. Ltd. |
| Phenoxyphenylacetic acid | U.S. Pat. No. 5,559,135, WO 9608487; |
| derivatives and analogues of | Merck & Co. Inc. |
| formula (I) and their salts | |
| 3-(and 5-) Benzene- | U.S. Pat. No. 5,514,696; Bristol-Myers |
| sulphonamido-isoxazole | Squibb Co. |
| derivatives of formula (I) | |
| and their salts | |
| Endothelin antagonists of | ZA 9500892; Abbott |
| formula (I) and their salts, | Laboratories |
| esters and prodrugs | |
| Phenoxyphenylacetic acid | U.S. Pat. No. 5,538,991, WO 9608486; |
| derivatives of formula (I) | Merck & Co. Inc. |
| and their salts | |
| N-Isoxazolyl-4;- | EP 702012; Bristol-Myers |
| heteroar(alk)yl-biphenyl-2- | Squibb Co. |
| sulphonamide derivatives of | |
| formula (I) and their | |
| enantiomers, diastereomers | |
| and salts | |
| Pyrrolidine and piperidine | U.S. Pat. No. 5,622,971, U.S. Pat. No. 5,731,434, U.S. Pat. No. |
| derivatives of formula (I) | 5,767,144, EP 776324; Abbott |
| and their salts | Laboratories |
| Peptide derivatives of | U.S. Pat. No. 5,550,110, EP 767801; |
| formula (I) and their salts | Warner Lambert Co. |
| Porphyrins of formula (I) or | JP 7330601; Kowa Co. Ltd. |
| their metal complexes or | |
| salts | |
| Triazine or pyrimidine | U.S. Pat. No. 5,840,722, EP 752854; BASF |
| derivatives of formula (I) | AG |
| Bicyclic piperazinone | DE 4341663; BASF AG |
| derivatives of formula (I) | |
| and their salts | |
| Benzenesulphonamide | U.S. Pat. No. 5,728,706, EP 658548; |
| derivatives of formula (I), | Tanabe Seiyaku Co. |
| and their salts, including 4- | |
| tert-butyl-N-(5-(4- | |
| methylphenyl)-6-(2-(5-(3- | |
| thienyl)pyrimidin-2- | |
| yloxy)ethoxy)pyrimidin-4-yl)- | |
| benzenesulphonamide | |
| RES-1214 of formula (I) | JP 7133254; Kyowa Hakko Kogyo |
| Bicyclic pyrimidine or 1,4- | U.S. Pat. No. 5,693,637, EP 733052, EP |
| diazepine derivatives of | 733052; BASF AG., Hoechst AG. |
| formula (I) and their acid | |
| addn. salts | |
| 5,11-Dihydro-11-oxo- | U.S. Pat. No. 5,420,123; Bristol-Myers |
| dibenzo(b,e) diazepine | Squibb Co. |
| derivatives of formula (I) | |
| Diaryl- and aryloxy compounds | U.S. Pat. No. 6,211,234, EP 728128; Rhone |
| of formula (I), their salts, | Poulenc Rorer Ltd. |
| N-oxides and prodrugs | |
| Non-peptidic compounds | U.S. Pat. No. 5,492,917, WO 9508989; |
| incorporating a cyclobutane | Merck & Co. Inc. |
| ring of formula (I) and their | |
| salts | |
| Amino acid derivatives of | WO 9508550; Abbott |
| formula (I) and their salts | Laboratories |
| Substituted 2(5H) furanone, | EP 714391; Warner Lambert Co. |
| 2(5H) thiophenone and 2(5H) | |
| pyrrolone derivatives of | |
| formula (I) and their salts | |
| Cyclopentene derivatives of | U.S. Pat. No. 5,714,479, EP 714897; Banyu |
| formula (I) and their salts | Pharm Co. Ltd. |
| Cyclopentane derivatives of | WO 9505372; Banyu Pharm Co. |
| formula (I) and their salts | Ltd. |
| Thienopyrimidine deriv. of | EP 640606; Takeda Chem. Ind. |
| formula (I) or one of its | Ltd., Takeda Pharm Ind. Co. |
| salts | Ltd. |
| Heteroaromatic ring-fused | U.S. Pat. No. 5389620, U.S. Pat. No. 5,714,479, EP |
| cyclopentene derivatives of | 714897; Banyu Pharm Co. Ltd. |
| formula (I), and their salts | |
| Phenalkyl substd. phenyl | U.S. Pat. No. 5,686,478, EP 710235; Merck |
| compounds of formula (I) and | & Co. Inc. |
| their salts | |
| Benzimidazolinone compounds | U.S. Pat. No. 5,391,566, WO 9503044; |
| substd. with | Merck & Co. Inc. |
| phenoxyphenylacetic acid | |
| derivatives of formula (I) | |
| and their salts | |
| Triterpene derivatives of | JP 6345716; Shionogi & Co. |
| formula (I) and their salts | Ltd. |
| N-Acyl-N-(amino- or hydroxy- | U.S. Pat. No. 5,888,972, EP 706532; |
| alkyl)-tripeptide derivatives | Fujisawa Pharm Co. Ltd. |
| of formula (I) and their | |
| salts | |
| Naphthalenesulphonamido- | U.S. Pat. No. 5,378,715; Bristol-Myers |
| isoxazoles of formula (I) and | Squibb Co. |
| their salts | |
| Amino acid phosphonic acid | U.S. Pat. No. 5,481,030, EP 639586; ADIR |
| derivatives of formula (I), | & CIE |
| their enantiomers, | |
| diastereoisomers, epimers and | |
| salts | |
| Endothelin antagonist of | U.S. Pat. No. 5,420,133; Merck & Co Inc |
| formula (I) or its salts | |
| Peptide derivatives for | WO 9419368; Banyu Pharm Co Ltd |
| formula (I) and their salts | |
| Endothelin antagonist of | U.S. Pat. No. 5,374,638; Merck & Co Inc. |
| formula (I) or its salts | |
| Compounds of formula (I), and | U.S. Pat. No. 5,352,800; Merck & Co. Inc. |
| their salts | |
| 1,4-Dihydro-4-quinolinones | U.S. Pat. No. 5,985,894, EP 498721; |
| and related compounds of | Roussel-Uclaf, Hoechst Marion |
| formula (I) and their isomers | Roussel |
| and salts | |
| Cyclic depsipeptide of | GB 2266890; Merck & Co. Inc. |
| formula (I) | |
| Condensed thiadiazole | U.S. Pat. No. 5,550,138, EP 562599; |
| derivatives of formula (I) | Takeda Chem. Ind. Ltd. |
| and their salts | |
| Compounds (I) and their | U.S. Pat. No. 5,550,138, EP 562599; |
| salts | Takeda Chem. Ind. Ltd. |
| Purified cyclic depsipeptide | U.S. Pat. No. 5,240,910; Merck & Co. Inc. |
| endothelin antagonist of | |
| formula (I) | |
| Cochinmycins (IV) and (V) | U.S. Pat. No. 5,240,910; Merck & Co. |
| | Inc. |
| Peptide derivatives (I) or | JP 5194592; Takeda Chem. Ind. |
| their salts | Ltd. |
| Cyclic peptides (I) or salts | JP 5194589; Takeda Chem. Ind. |
| thereof | Ltd. |
| Peptides of formula (I) and | U.S. Pat. No. 5,614,497, EP 552489; |
| their salts | Takeda Chem. Ind. Ltd. |
| Cyclic hexapeptide | EP 552417; Takeda Chem. Ind. |
| derivatives of formula (I) | Ltd. |
| and their salts, including | |
| cyclo-(D-Asp-Trp-Asp-D-Leu- | |
| Leu-D-Trp) (Ia) | |
| Indane and indene derivatives | EP 612244; Smithkline Beecham |
| of formula (I) and their | Corp. |
| salts | |
| Cyclic peptide derivatives of | U.S. Pat. No. 5,616,684, U.S. Pat. No. 5,883,075, EP |
| formula (I) and their salts | 528312; Takeda Chem. Ind. Ltd. |
| Endothelin (ET) analogue | U.S. Pat. No. 5,352,659, EP 499266; |
| peptides of formula (I) and | Takeda Chem. Ind. Ltd. |
| their salts | |
| Cyclic depsipeptides of | EP 496452, U.S. Pat. No. 4,810,692; Merck |
| formula (A) | & Co. Inc. |
| N-((2′-(((4,5-dimethyl-3- | U.S. Pat. No. 6,043,265; Bristol-Myers |
| isoxazolyl)amino)sulfonyl)-4- | Squibb Co. |
| (2-oxazolyl)(1,1′-bi | |
| phenyl)-2-yl)methyl)-N,3,3- | |
| trimethylbutanamide and salts | |
| thereof | |
| N-(4,5-dimethyl-3- | U.S. Pat. No. 6,043,265; Bristol-Myers |
| isoxazolyl)-2′-((3,3- | Squibb Co. |
| dimethyl-2-oxo-1- | |
| pyrrolidinyl)methy 1)-4′-(2- | |
| oxazolyl)(1,1′-biphenyl)-2- | |
| sulfonamide, and salts | |
| thereof. | |
| Substituted biphenyl | U.S. Pat. No. 5,780,473; Abbott |
| sulfonamide compounds of | Laboratories |
| formula (I), their | |
| enantiomers and | |
| diastereomers, and | |
| pharmaceutically acceptable | |
| salts thereof | |
| Compounds of formula (I) and | U.S. Pat. No. 6,162,927; Abbott |
| salts thereof, including | Laboratories |
| intermediates in the process | |
| of preparation | |
| Heterocyclyl-substituted | U.S. Pat. No. 5,780,473 |
| biphenylsulfonamide | |
| Crystalline sodium salt of 2- | WO 2001030767; BASF AG |
| pyrimidinyloxy-3,3- | |
| diphenylpropionic acid | |
| derivative | |
| Phenyl compounds substituted | U.S. Pat. No. 6,124,343; Rhone-Poulenc |
| with heteroaryl (preferably | Rorer Ltd. |
| thienyl methoxy) moieties and | |
| their derivatives | |
| 1,3-benzodioxole compounds | U.S. Pat. No. 6,048,893; Rhone-Poulenc |
| | Rorer Ltd. |
| Biphenyl sulfonamides of | U.S. Pat. No. 1998-91847P, EP 1094816; |
| formula (I) | Bristol-Myers Squibb Co. |
| Compound (I) or its salt | EP 950418; Takeda Chem Ind |
| | Ltd. |
| A carboxylic acid of formula | EP 1014989; Knoll AG |
| (I) or (II), including s- | |
| triazinyl-or pyrimidinyl- | |
| substituted alkanoic acid | |
| derivative | |
| Endothelin antagonist of | AU 739860; Knoll AG |
| formula (I) | |
| N-(3,4-dimethyl-5- | U.S. Pat. No. 5,916,907, U.S. Pat. No. 5,612,359; |
| isoxazolyl)-4-(2-oxazolyl)(1, | Bristol-Myers Squibb Co. |
| 1′-biphenyl)-2- | |
| sulphonamide and its salts | |
| N-((2′-(((4,5-dimethyl-3- | U.S. Pat. No. 5,916,907, U.S. Pat. No. 5,612,359; |
| isoxazolyl) amino)sulphonyl)- | Bristol-Myers Squibb Co. |
| 4-(2-oxazolyl) (1,1′- | |
| biphenyl)-2-yl)methyl)- | |
| N,3,3-trimethyl butanamide | |
| and its salts | |
| Pyrrolidine derivatives of | U.S. Pat. No. 1997-794506, EP 885215; |
| formula (I) and their salts, | Abbott Laboratories |
| including (2R,3R,4S)-2-(3- | |
| fluoro-4-methoxyphenyl)-4- | |
| (1,3-benzodioxol-5-yl)-1-(2- | |
| (N-propyl-N- | |
| pentanesulphonylamino)ethyl)- | |
| pyrrolidine-3-carboxylic acid | |
| Phenoxyphenylacetic acids and | U.S. Pat. No. 5,565,485; Merck & Co., |
| derivatives of the general | Inc. |
| structural formula I | |
| Compounds of the formula I, | U.S. Pat. No. 5,641,793; Zeneca Limited |
| namely novel pyridine | |
| derivatives including N-(2- | |
| pyridyl)sulphonamides, and | |
| pharmaceutically-acceptable | |
| salts thereof | |
| N-heterocyclic sulfonamides | U.S. Pat. No. 5,668,137; Zeneca Ltd. |
| of the formula I, their | |
| pharmaceutically-acceptable | |
| salts, and pharmaceutical | |
| compositions containing them | |
| Phenoxyphenylacetic acids and | U.S. Pat. No. 5,668,176; Merck & Co. |
| derivatives of the general | Inc. |
| structural formula I | |
| Compounds of Formula I and | U.S. Pat. No. 5,691,373; Warner-Lambert |
| the pharmacologically | Company |
| acceptable salts thereof, | |
| including 2-benzo- | |
| >1,3dioxol-5-yl-4-(4- | |
| methoxyphenyl)-4-oxo-3- | |
| (3,4,5-trimethoxybenzyl)-but- | |
| 2-enoic acid | |
| Phenoxyphenylacetic acids and | U.S. Pat. No. 5,767,310; Merck & Co., |
| derivatives of general | Inc. |
| structural formula (I) | |
| N-heterocyclyl sulphonamide | U.S. Pat. No. 5,861,401, U.S. Pat. No. 6,083,951; |
| derivatives and their | Zeneca Limited |
| pharmaceutically acceptable | |
| salts | |
| Heterocyclic compounds of the | U.S. Pat. No. 5,866,568; Zeneca Limited |
| formula I and salts thereof, | |
| including N-heterocyclyl | |
| sulphonamides | |
| Pyrimidines of formula I | U.S. Pat. No. 5,883,254, 6,121,447, |
| | 6,274,734; Hoffmann-La Roche |
| | Inc. |
| Nonpeptide compounds of | U.S. Pat. No. 6,017,916; Warner-Lambert |
| formula I | Company |
| Ketoacid compounds of the | U.S. Pat. No. 6,043,241; Warner-Lambert |
| formula I and | Company |
| pharmaceutically acceptable | |
| salts thereof. | |
| 1,2-diheteroethylene | U.S. Pat. No. 6,136,971; Roche Colorado |
| sulfonamides | Corporation |
| Compound of the formula (I) | U.S. Pat. No. 6,218,427; Shionogi & Co., |
| and salts or hydrates thereof | Ltd. |
| Peptides of the formula (I) | U.S. Pat. No. 6,251,861; Takeda Chemical |
| and their salts | Industries, Ltd. |
| Substituted pyrazin-2-yl- | U.S. Pat. No. 6,258,817; Zeneca Ltd. |
| sulphonamide (-3-pyridyl) | |
| compounds of formula I, | |
| salts, and pharmaceutical | |
| compositions containing them. | |
| 4,5-Dihydro-(1H)- | U.S. Pat. No. 6,291,485; Teikoku Hormone |
| benz(g)indazole-3-carboxylic | Mfg. Co., Ltd. |
| acid derivatives of formula I | |
| and their salts | |
| Nonpeptide endothelin I | U.S. Pat. No. 6,297,274; Warner-Lambert |
| antagonists of formula I | Company |
| Carboxylic acid derivatives | EP 946524; BASF AG |
| of formula (I) and their | |
| salts, enantiomers and | |
| diastereomers | |
| 4′-Heterocyclyl(alkyl)-N- | U.S. Pat. No. 5,846,990; BRISTOL-MYERS |
| isoxazolyl-biphenyl-2-yl | SQUIBB CO |
| sulphonamides of formula | |
| (I), and their enantiomers, | |
| diastereoisomers, and salts | |
| Biphenyl sulfonamides of | WO 200001389; BRISTOL-MYERS |
| formula (I) | SQUIBB CO |
| Endothelin antagonist of | WO 9916444, EP 1019055; KNOLL |
| formula (I) | AG |
| Endothelin antagonist of | DE 19743140; KNOLL AG |
| formula (I) | |
| Pyrrolidine derivatives of | WO 9730045; ABBOTT |
| formula (I) and their salts | Laboratories |
| Canrenoate Potassium | U.S. Pat. No. 5,795,909 |
| Canrenone | U.S. Pat. No. 5,795,909 |
| Dicirenone | U.S. Pat. No. 5,795,909 |
| Mexrenoate Potassium | U.S. Pat. No. 5,795,909 |
| Prorenoate Potassium | U.S. Pat. No. 5,795,909 |
| 4-amino-5-furyl-2-yl-4H- | Chinese Chemical Letters |
| 1,2,4-triazolethiol | (2003), 14(8), 790-793. |
| derivatives | |
| 3-alkylthio-4-arylideneamino- | Chinese Chemical Letters |
| 5-(2-furyl)-1,2,4-triazole | (2003), 14(8), 790-793. |
| derivatives | |
| BMS-346567 | Abstracts of Papers, 226th ACS |
| | National Meeting, New York, |
| | NY, Sep. 7-11, 2003 |
| | (2003), MEDI-316.; Bristol- |
| | Myers Squibb |
| Alkanesulfonamides of formula I | WO2003055863 |
| Benzo-fused heterocycles of | WO 2003013545 |
| formula I | |
| (S*)-(4,6-dimethylpyrimidin- | WO 2003013545 |
| 2-yloxy)-[(5S*)-2-oxo-5- | |
| phenyl-1-(2,4,6- | |
| trifluorobenzyl)-2,3,4,5- | |
| tetrahydro-1H-benzo[e][1, | |
| 4]diazepin-5-yl]acetic | |
| acid | |
| (S*)-(3,5- | WO 2003013545 |
| dimethoxyphenoxy)[(1S*)-1- | |
| phenyl-1,2,3,4- | |
| tetrahydroisoquinolin-1-yl]acetic | |
| acid | |
| N-phenylimidazole derivatives | U.S. Pat. No. 2003004202; U.S. Pat. No. 2003153567; |
| | U.S. Pat. No. 6,620,826 |
| Pyrimidine-sulfamides of | WO 2002053557 |
| formula I | |
| Arylalkylsulfonamides of | WO 2002024665 |
| formulas I and II | |
| Pyrimidino-pyridazines of | U.S. Pat. No. 2002061889; U.S. Pat. No. 6,670,362 |
| formulas I and II | |
| Arylethenesulfonic acid | U.S. Pat. No. 2003220359 |
| pyrimidinylamides of formula I | |
| Mercaptopyrrolidine | U.S. Pat. No. 2002049243; U.S. Pat. No. 6,541,638 |
| carboxamides related | |
| compounds of formula I | |
| (2S,4R)-4-mercapto-1- | U.S. Pat. No. 2002049243; U.S. Pat. No. 6,541,638 |
| (naphthalene-2- | |
| sulfonyl)pyrrolidine-2- | |
| carboxylic acid methyl(o- | |
| totylcarbamoylmethyl)amide | |
| N-aminocarbonyl-β-alanines of | WO 2001090079 |
| formula I | |
| 4-(4-pyrimidinyloxy)-2-butyn- | U.S. Pat. No. 2003087920 |
| 1-ol derivatives of formulas | |
| I and II | |
| Pyrimidinyloxypropionates of | WO 2001005771 |
| formula I | |
| (S)-2-(4-methoxy-5- | WO 2001005771 |
| methylpyrimidin-2-yloxy)-3- | |
| methoxy-3,3-diphenylpropionic | |
| acid | |
| 2-pyrimidinyloxypropanoates | WO 2000073276 |
| and analogs thereof of | |
| formulas I and II | |
| Pyrrolidinecarboxylates of | U.S. Pat. No. 6,124,341 |
| formulas I and II | |
| N-(pyridylpyrimidinyl)heterocyclysulfonamides | U.S. Pat. No. 6,417,360 |
| 4-(heterocyclylsulfonamido)- | U.S. Pat. No. 6,242,601 |
| 5-(2-methoxyphenoxy)-2-phenyl | |
| derivatives of formula I | |
| Pyridylpyrimidines of formula I | U.S. Pat. No. 6,242,601 |
| Monoargininyl salts | U.S. Pat. No. 6,300359 |
| (E)-3-[1-n-butyl-5-[2-(2- | U.S. Pat. No. 6,300359 |
| carboxyphenyl)methoxy-4- | |
| chlorophenyl]-1H-pyrazol-4- | |
| yl]-2-[(5-methoxy-2,3- | |
| dihydrobenzofuran-6- | |
| yl)methyl]-prop-2-enoic acid | |
| 3-carbamoylalkoxy-2- | U.S. Pat. No. 6,509,341 |
| aryloxypropionates and | |
| analogs thereof of formula I | |
| Indole derivatives of | U.S. Pat. No. 6,017,945; U.S. Pat. No. 6,136,843; U.S. Pat. No. |
| formula I | 6,306,852; U.S. Pat. No. 2001014677; U.S. Pat. No. |
| | 6,384,070 |
| α-hydroxy acid derivatives of | U.S. Pat. No. 6,686,369 |
| formula I | |
| 4-benzodioxolylpyrrolidine-3- | WO 9730046 |
| carboxylates and analogs | |
| thereof of formula I | |
| Isoxazoles and imidazoles of | U.S. Pat. No. 6,030,970; U.S. Pat. No. 6,174,906 |
| formula I | |
| Furan and thiophene | U.S. Pat. No. 6,017,952; U.S. Pat. No. 6,051,599 |
| derivatives of formulas I and II | |
| N-isoxazolylthiophenesulfon- | U.S. Pat. No. 5,490,962; U.S. Pat. No. 5,518,680; U.S. Pat. No. |
| amides and analogs thereof of | 5,594,021; U.S. Pat. No. 5,962,490; U.S. Pat. No. |
| formulas I and II | 6,139,574; U.S. Pat. No. 6,342,610; U.S. Pat. No. |
| | 6,331,637; U.S. Pat. No. 6,514,518; U.S. Pat. No. |
| | 6,632,829 |
| N-isoxazolyl(hetero) | U.S. Pat. No. 5,571,821; U.S. Pat. No. 5,490,962; U.S. Pat. No. |
| arenesulfonamides of formulas | 5,464,853; U.S. Pat. No. 5,514,691; U.S. Pat. No. |
| I and II | 5,518,680; U.S. Pat. No. 5,591,761; U.S. Pat. No. |
| | 5,594,021; U.S. Pat. No. 5962,490; U.S. Pat. No. |
| | 6,030,991; U.S. Pat. No. 6,139,574; U.S. Pat. No. |
| | 6,331,637; U.S. Pat. No. 6,376,523; U.S. Pat. No. |
| | 6,541,498; U.S. Pat. No. 6,514,518; U.S. Pat. No. |
| | 6,613,804 |
| N-(4-pyrimidinyl)sulfonamides | EP 713875 |
| of formula I | |
| Arylimidazolylpropenoates and | U.S. Pat. No. 2003153567; U.S. Pat. No. 6,620,826 |
| related compounds of formula I | |
| (E)-3-[s-butyl-1-[2-[N- | U.S. Pat. No. 2003153567; U.S. Pat. No. 6,620,826 |
| (phenylsulfonyl)]carboxamido- | |
| 4-methoxyphenyl]-1H-imidazol- | |
| 5-yl]-2-[(2-methoxy-4,5- | |
| methylenedioxyphenyl)methyl]- | |
| 2-propenoic acid dipotassium | |
| salt | |
| Pyrimidine and triazine | U.S. Pat. No. 5,932,730; U.S. Pat. No. 6,197,958; U.S. Pat. No. |
| derivatives of formulas I and | 6,600,043 |
| II | |
| Indane and Indene derivatives | U.S. Pat. No. 6,271,399; U.S. Pat. No. 6,087,389; U.S. Pat. No. |
| of formula I | 6,274,737; U.S. Pat. No. 2002002177; U.S. Pat. No. |
| | 6,448,260 |
| Heteroaromatic ring-fused | U.S. Pat. No. 5,389,620; U.S. Pat. No. 5,714,479 |
| cyclopentene derivatives of | |
| formula I | |
| (5RS,6SR,7RS)-6-carboxy-7-(4- | U.S. Pat. No. 5,389,620; U.S. Pat. No. 5,714,479 |
| methoxyphenyl)-5-(3,4- | |
| methylenedioxyphenyl)cyclopenteno[1, | |
| 2]-bpyridine | |
| Pyrido[2,3-d]pyrimidinesof | U.S. Pat. No. 5,654,309 |
| formulas I and II | |
| Pyrido[2,3-d]pyrimidine-3- | U.S. Pat. No. 5,654,309 |
| acetic acid of formula II | |
| 4-Heterocyclyl-sulfonamidyl- | WO 200052007 |
| 6-methoxy-5-(2- | |
| methoxyphenoxy)-2-pyridyl- | |
| pyrimidine derivatives of | |
| formula I | |
| Alpha-hydroxy-carboxylic acid | DE 19614533 |
| derivatives of formula I | |
| 2-(4,6-dimethylpyrimidin-2- | DE 19614533 |
| yloxy)-3,3-diphenylbutyric | |
| acid | |
| 2-formylaniline derivatives | WO 2003080643 |
| of formula V | |
| 6a-{3-[2-(3-carboxy- | WO 2003080643 |
| acryloylamino)-5- | |
| hydroxyphenyl]- | |
| acryloyloxymethyl}- | |
| 2,2,6b,9,9,12a-hexamethyl-10- | |
| oxo1,3,4,5,6,6a,7,8,8a,9,9,12a, | |
| 12b,13,14b-octadecahydro- | |
| 2H-picene-4a-carboxylic acid | |
| or its salts | |
| Alkanesulfonamides of | WO 2003055863 |
| formulas I or Ia | |
| ethanesulfonic acid {6-[2-(5- | WO 2003055863 |
| bromo-pyrimidin-2-yloxy)- | |
| ethoxy]-5-para-tolyl- | |
| pyrimidin-4-yl}-amine | |
| N-phenyl imidazole | U.S. Pat. No. 2003004202 |
| derivatives of formula I or | |
| salts thereof | |
| (E)-3-[2-butyl-1-[2-(2- | U.S. Pat. No. 2003004202 |
| carboxyphenyl)methoxy-4- | |
| methoxy]phenyl-1H-imidazol-5- | |
| yl]-2-[(2-methoxy-4,5- | |
| methylenedioxyphenyl)methyl]- | |
| 2-propenoic acid | |
| Benmzofused heterocycle | WO 2003013545 |
| derivatives of formula I and | |
| salts thereof | |

Also included in Table 1 are the following ERA's:

Atrasentan, avosentan, tezosentan, clazosentan and propyl-sulfamic acid {5-(4-bromo-phenyl)-6-[2-(5-bromo-pyrimidin-2-yloxy)-ethoxy]-pyrimidine-4-yl}-amide.

The amount of endothelin receptor antagonist that is administered and the dosage regimen for the methods of this invention also depend on a variety of factors, including the age, weight, sex and medical condition of the subject, the severity of the pathological condition, the route and frequency of administration, and the particular endothelin receptor antagonist employed, and thus may vary widely. A daily dose administered to a subject of about 0.001 to 100 mg/kg body weight, or between about 0.005 and about 60 mg/kg body weight, or between about 0.01 and about 50 mg/kg body weight, or between about 0.015 and about 15 mg/kg body weight, or between about 0.05 and about 30 mg/kg body weight, or between about 0.075 to 7.5 mg/kg body weight, or between about 0.1 to 20 mg/kg body weight, or between about 0.15 to 3 mg/kg body weight, may be appropriate.

The amount of endothelin receptor antagonist that is administered to a human subject typically will range from about 0.1 to 2400 mg, or from about 0.5 to 2000 mg, or from about 0.75 to 1000 mg, or from about 1 mg to 1000 mg, or from about 1.0 to 600 mg, or from about 5 mg to 500 mg, or from about 5.0 to 300 mg, or from about 10 mg to 200 mg, or from about 10.0 to 100 mg. The daily dose can be administered in one to six doses per day.

In a preferred embodiment, bosentan is administered at a daily dose to a subject of about 62.5 mg twice a day, or 125 mg twice a day to adult patients.

The endothelin receptor antagonists and their pharmaceutically usable salts can be used as medicament (e.g. in the form of pharmaceutical preparations). The pharmaceutical preparations can be administered internally, such as orally (e.g. in the form of tablets, coated tablets, dragees, hard and soft gelatine capsules, solutions, emulsions or suspensions), inhalations, nasally (e.g. in the form of nasal sprays) or rectally (e.g. in the form of suppositories). However, the administration can also be effected parenterally, such as intramuscularly or intravenously (e.g. in the form of injection solutions).

The endothelin receptor antagonists and their pharmaceutically usable salts can be processed with pharmaceutically inert, inorganic or organic adjuvants for the production of tablets, coated tablets, dragees, and hard gelatine capsules. Lactose, corn starch or derivatives thereof, talc, stearic acid or its salts etc. can be used, for example, as such adjuvants for tablets, dragees, and hard gelatine capsules.

Suitable adjuvants for soft gelatine capsules, are, for example, vegetable oils, waxes, fats, semi-solid substances and liquid polyols, etc. Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils.

Suitable adjuvants for suppositories are, for example, natural or hardened oils, waxes, fats, semi-solid or liquid polyols.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

Experimental Section/Biology:

The findings with bosentan can be extrapolated to other endothelin receptor antagonists as mentioned above, because endothelin-1 (ET-1) has been shown to play a central role in the development of fibrosis and therefore drugs used to target and inhibit the action of ET-1 will be effective in treating early fibrosis.

Indeed, at a whole body level, transgenic mice overexpressing ET-1 develop a phenotype of fibrosis (pulmonary and renal). This fibrosis is a direct consequence of ET-1 action, because there is no associated increase in blood pressure (1, 2). At a cellular and biochemical level also, endothelin is a central mediator of fibrosis (3). ET-1 induces chemotaxis and proliferation of fibroblasts, increases the synthesis and production of various extracellular matrix proteins like laminin, collagen, and fibronectin, while inhibiting collagenase activity. ET-1 also induces expression of other profibrotic factors, such as connective tissue growth factor and transforming growth factor beta (TGF-β). ET-1 also increases the pro-inflammatory effector, nuclear factor-kappa B (NF-κB). In a rat lung model of fibrosis (bleomycin-induced) there was an elevation of ET-1 levels prior to an increase in collagen content which, along with its localization within developing fibrotic lesions, provides further evidence of a pro-fibrotic role for ET-1 at an early stage in the pathogenesis of bleomycin-induced lung fibrosis (20).

Bosentan, by antagonizing the profibrotic properties of ET-1, prevents initiation of fibrosis (3). Bosentan in cell cultures decreases collagen synthesis, increases collagenase expression, inhibits extracellular matrix deposition (4) and reduces NF-κB expression (5). Consequently bosentan in vivo is a potent anti-fibrotic agent in various animal models of fibrosis (6-11).

Since ET-1 is a central player of fibrosis, the findings with bosentan can be extrapolated to all other antagonists of endothelin receptors. For example, in cell cultures, bosentan and another endothelin receptor antagonist, PD 156707, attenuated fibroblast proliferation induced by ET-1 in human fibroblasts (12), increased matrix metalloprotease-1 (collagenase) production (4), and reduced the ability to contract a collagen matrix (13). Another endothelin receptor antagonist, BQ-123, decreased fibronectin synthesis induced by ET-1 or angiotensin II in rat mesangial cells (14). Another antagonist, PED-3512-PI, increased collagenase activity induced by ET-1 and ET-3 in rat cardiac fibroblasts (15).

In in vivo models of fibrosis, the endothelin receptor antagonist FR1 39317 attenuated the expression of collagen, laminin and TGF-β mRNA in diabetic rat kidney (16). Darusentan decreased the accumulation of collagen in norepinephrine -induced aortic remodeling and fibrosis (17). Other endothelin receptor antagonists decreased cardiac fibrosis in heart failure and hypertension models (18, 19).

Experimental Setup for the Evaluation of the Antifibrotic Properties of Bosentan and of other Endothelin Receptor Antagonists

Experiments were performed on the mouse embryonic fibroblast cell line Swiss 3T3 (Deutsche Sammlung fur Mikroorganismen und Zellen, DSMZ ACC 173). Cells were starved for 24 h in serum-free medium or medium containing 0.5% serum followed by a 24 h incubation with endothelin-1 at a concentration giving approximately 50% or preferably 80% of its maximal efficacy, in presence either of vehicle or of an antagonist at increasing concentrations or an antagonist in combination with Pirfenidone.

Potential cytotoxic effects are excluded by assessing fibroblast proliferation using the MTS reagent (21). Collagen neo-synthesis by fibroblasts is assessed by measuring ³H-proline incorporation (22).

Several endothelin receptor antagonists have been tested according to the above-mentioned experimental method.

### Experimental Results:

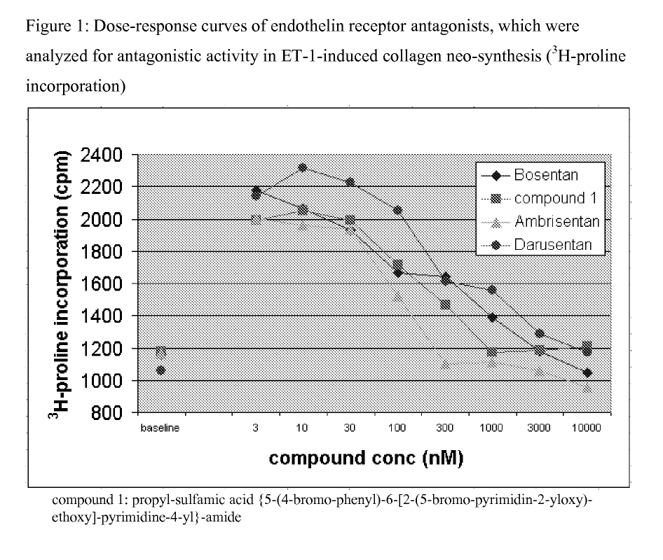

In this cell culture model of early fibrosis using Swiss 3T3 mouse embryonic fibroblasts, the concentration-dependent effect of ET-1 on collagen neo-synthesis was measured, and yielded an EC₅₀ (concentration of ET-1 giving 50% of maximal effect) of 0.24 nM. Using a concentration of ET-1 of 1 nM (EC₈₀), the below mentioned endothelin receptor antagonists were analyzed for antagonistic activity on ET-1-induced collagen neo-synthesis. FIG. 1 shows representative dose-response curves for a selection of tested compounds. The summary for seven tested endothelin receptor antagonists is presented in table 2.

We conclude that all tested antagonists fully antagonize ET-1 -induced collagen neo-synthesis to baseline values, with IC₅₀ values ranging from 59 nM to 369 nM.

TABLE 2
IC₅₀ values of different ERAs on ET-1-induced
collagen neo-synthesis in 3T3 fibroblasts (n >= 2)

| Compound | IC₅₀ (nM) |
|---|---|
| Bosentan | 214 |
| Compound 1 | 114 |
| Ambrisentan | 79 |
| Darusentan | 221 |
| TBC3711 | 59 |
| Sitaxsentan | 369 |
| Avosentan | 330 |

Compound 1 = propyl-sulfamic acid {5-(4-bromo-phenyl)-6-[2-(5-bromo-pyrimidin-2-yloxy)-ethoxy]-pyrimidine-4-yl}-amide

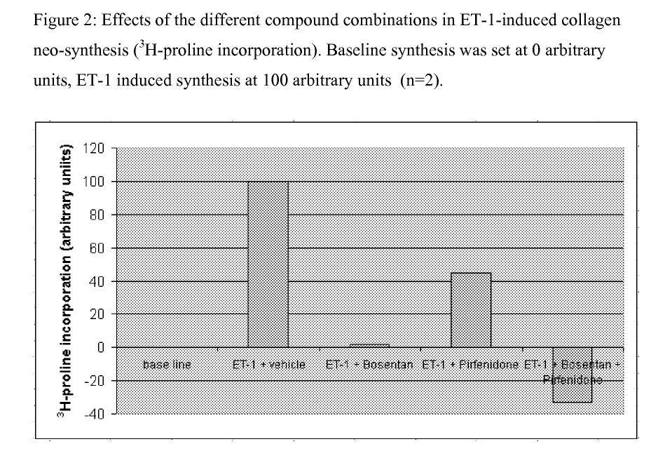

Next, the combination of pirfenidone (Sigma P-2116) and bosentan in antagonizing ET-1-induced collagen neo-synthesis was tested. To this end, fibroblasts were treated with either vehicle, bosentan (1 μM), pirfenidone (1 mM) or a combination of bosentan and pirfenidone for 24 h followed by the determination of collagen neo-synthesis. FIG. 2 shows the effects of the different compound combinations in ET-1 -induced collagen neo-synthesis.

The results show that 1 μM bosentan alone reverses ET-1 -induced collagen synthesis to baseline while pirfenidone alone has a 55% inhibitory effect on collagen neo-synthesis. Combination of both compounds has an additive effect on collagen neo-synthesis leading to a 33% drop below the value of baseline synthesis.

Clinical Evidence

BUILD 1 study was a multicentric, randomized, double-blind, placebo-controlled, phase II/III study in IPF patients. The aim of this study was to demonstrate that bosentan improves the exercise capacity of patients with IPF as assessed by the 6-minute walk test (6MWT) distance. The secondary objectives of the study were to demonstrate that bosentan delays time to death or treatment failure, improves pulmonary function tests (PFTs), dyspnea and quality of life and is safe and well tolerated in this patient population. Treatment failure was defined either as worsening of PFTs or the occurrence of an acute decompensation of IPF. PFT worsening was defined as 2 out of the following 3 criteria
Decrease from baseline ≧10% in Forced vital capacity (FVC)
Decrease from baseline ≧15% in diffusion capacity for carbon monoxide (DLCO).
Decrease from baseline ≧4% in O2 saturation (blood gas) at rest or increase from baseline ≧8 mmHg in alveolar capillary O2 gradient (A-a PO2).

Main inclusion criteria: proven IPF diagnosis <3 years duration, either via a surgical lung biopsy or when not done according to the ATS/ERS consensus criteria (see above). The main inclusion criteria were the presence of FVC ≧50% of predicted value and DLCO ≧30% of predicted value.

A total of 158 patients were randomly allocated to treatment with bosentan (n=74) or placebo (n=84). Overall, 154 randomized patients received at least one dose of study medication and had at least one valid post baseline value for the primary endpoint (n=71 on bosentan, n=83 on placebo). Following a screening period (≦4 weeks), eligible patients were randomized to either bosentan or placebo (1:1), started on oral bosentan 62.5 mg b.i.d. or matching placebo, and up-titrated at Week 4 to achieve the target dose (125 mg b.i.d. or matching placebo) for the remainder of the treatment Period unless down-titrated for reasons of tolerability. The planned treatment period 1 was 12 months. Patients were evaluated at regular interval up to End-of-Period 1 (Month 12 months) and up to the End-of-Study i.e. when the last patient has his/her last visit. The 6MWT and pulmonary function tests were evaluated at each visit.

The All-Treated set of patients included 154 randomized patients who had received at least one dose of study medication and had at least one valid post baseline value for the primary endpoint (n=71 on bosentan, n=83 on placebo). The treatment groups were generally well matched with regard to demographics and baseline disease characteristics.

Although bosentan did not show improvement in the primary endpoint of the 6MWT at the End-of-Period 1, BUILD-1 showed a positive and clinically relevant trend for the efficacy of bosentan in prevention of clinical worsening. The most important clinical finding was a trend for a treatment effect on the PFT score defined as either the occurrence of death or treatment failure (worsening of PFTs or acute respiratory decompensation) at the End-of-Period 1, which was a pre-defined secondary endpoint, (22.5% in the bosentan group compared to 36.1%, in the placebo group corresponding to a relative risk ratio of 0.62, p=0.0784). PFT scoring was mainly driven by the change in FVC and DLCO.

Post hoc subpopulation analyses were undertaken to determine which population would best show a treatment effect on PFT scores. Age, gender, site location, baseline walk tests or pulmonary function tests were not predictive of any particular treatment effect with bosentan. Surprisingly, as can be seen in Table 3, the 99 patients who had a surgical lung biopsy to establish the IPF diagnosis showed a dramatic statistically significant treatment effect with a relative risk ratio of 0.32, (95% confidence interval (CI) 0.14-0.74).

TABLE 3
Produced by sturlor on 31MAR06 - Data dump of 14DEC05
Ro 47-0203, Protocol: AC-052-320
Table PFTP_EOP1_BIO_T: PFTs scores at end of period 1
Analysis set: All treated - Patients with surgical lung biopsy performed

| | Placebo | Bosentan |
|---|---|---|
| | N = 50 | N = 49 |
| n | 50 | 49 |
| Worsened | 19 (38.0%) | 6 (12.2%) |
| 95% confidence limits | 24.7%, 52.8% | 4.6%, 24.8% |
| Treatment effect: | | |
| Relative risk | | 0.32 |
| 95% confidence limits | | 0.14, 0.74 |
| p-value Fisher's exact test | | 0.0050 |
| n | 50 | 49 |
| Improved | 0 (0.0%) | 2 (4.1%) |
| 95% confidence limits | 0.0%, 7.1% | 0.5%, 14.0% |
| Treatment effect: | | |
| Relative risk | | |
| 95% confidence limits | | |
| p-value Fisher's exact test | | 0.2424 |
| (Page 1/1) | | |

In contrast, the 58 patients who were diagnosed without a surgical lung biopsy (SLB) showed no treatment effect (relative risk ratio of 1.36, 95% CI 0.70-2.65). Whether this observation was simply due to a chance finding could only be determined by comparing the baseline characteristics of those 2 subgroups of patients.

As seen on Table 4 the only obvious difference was that the non-SLB patients were older than the SLB patients. There were no parameters of the lung function tests suggesting that one group had a more advanced disease than the other.

TABLE 4

| | SLB diagnosis | | Non SLB diagnosis | |
|---|---|---|---|---|
| | Placebo | Bosentan | Placebo | Bosentan |
| | N = 50 | N = 49 | N = 34 | N = 24 |
| Sex male (%) | 80 | 64 | 67.6 | 70.8 |
| Age mean (yrs) | 62.4 | 64.1 | 69 | 68.8 |
| 41-60 years | 40.0 | 22.0 | 17.6 | 12.5 |
| (%) | | | | |
| 61-70 yrs (%) | 38 | 52 | 35.3 | 41.7 |
| >70 yrs (%) | 22.0 | 24.0 | 47.1 | 45.8 |
| Weight (kg) | 88.5 | 87 | 77 | 80.1 |
| Race (white %) | 90 | 92 | 94.1 | 91.7 |
| Location (% US) | 64 | 72 | 67.6 | 45.8 |
| Duration IPF | 2.4 | 2.2 | 2.6 | 2.7 |
| symptoms (yrs) | | | | |
| FVC (%) | 67.4 | 67.1 | 72.8 | 65.4 |
| DIco (%) | 41.7 | 43.7 | 40.9 | 40.8 |
| TLC (%) | 65.1 | 64.1 | 67.7 | 66.0 |
| RV (%) | 59.6 | 58 | 64 | 65.6 |
| FEV1(%) | 78.9 | 78.7 | 86.6 | 81.5 |

Yrs years, % percent of predicted value;
TLC total lung capacity;
RV residual volume;
FEV1 forced expiratory volume in 1 sec

As seen on Table 5 the only obvious difference was that the non-SLB patients were older than the SLB patients. The lung function tests were well balanced between the 2 groups.

TABLE 5

| | Biopsy diagnosis* | | CT diagnosis | |
|---|---|---|---|---|
| | Placebo | Bosentan | Placebo | Bosentan |
| A | N = 50 | N = 50 | N = 34 | N = 24 |
| Sex male (%) | 80 | 64 | 67.6 | 70.8 |
| Age mean (yrs) | 62.4 | 64.1 | 69 | 68.8 |
| 41-60 years (%) | 40.0 | 22.0 | 17.6 | 12.5 |
| 61-70 yrs (%) | 38 | 52 | 35.3 | 41.7 |
| >70 yrs (%) | 22.0 | 24.0 | 47.1 | 45.8 |
| Weight (kg) | 88.5 | 87 | 77 | 80.1 |
| Race (white %) | 90 | 92 | 94.1 | 91.7 |
| Location (% US) | 64 | 72 | 67.6 | 45.8 |
| Duration IPF symptoms | 2.5 | 2.4 | 2.6 | 2.7 |
| (yrs) | | | | |
| FVC (%) | 67.4 | 67.1 | 72.8 | 65.4 |
| DIco (%) | 41.7 | 43.7 | 40.9 | 40.8 |
| TLC (%) | 65.1 | 64.0 | 67.7 | 66.0 |
| RV (%) | 59.6 | 58 | 64 | 65.6 |
| FEV₁(%) | 78.9 | 78.7 | 86.6 | 81.5 |

*Safety population for which one bosentan patient did not have a post baseline efficacy assessment
Yrs years, % percent of predicted value;
TLC total lung capacity;
RV residual volume;
FEV1 forced expiratory volume in 1 sec

The only remaining logical explanation was that these 2 groups differed in their HRCT at presentation. Before undertaking a central reading of all available CTs, the following hypothesis was built.

Three possible explanations were tested why patients with SLBs would have had a better treatment effect than those without:
Patients with surgical lung biopsy had little or no honeycombing
Patients with surgical lung biopsy had less extensive fibrosis, and therefore more difficult to make a confident CT diagnosis
Patients with surgical lung biopsy had substantially more ground-glass abnormality than the others
   With these in mind, we formulated the following hypotheses:

Extent of honeycombing in IPF is a predictor of non-response to treatment.

Extent of ground-glass abnormality is a predictor of response to treatment

The analyses were run by a single radiologist who was blinded to the group allocation. Each patient CT was scored for honeycomb as well as ground-glass from the 3 zones of each lung namely upper mid and lower zone. Increment for HC and ground-glass was rounded to the upper 5%.

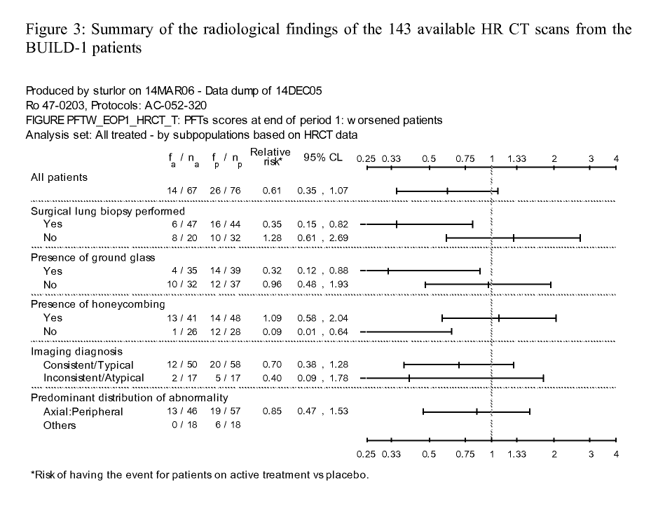

FIG. 3 summarizes the radiological findings of the 143 available HRCT scans from the BUILD-1 patients. Irrespective of the need for SLB for establishing the diagnosis of IPF the pre-specified hypothesis was verified that the presence of ground-glass or the absence of honeycomb were strong predictors of a treatment effect with bosentan as well as the predominant distribution of abnormality (sub-pleural vs. diffuse or axial peripheral vs. others).

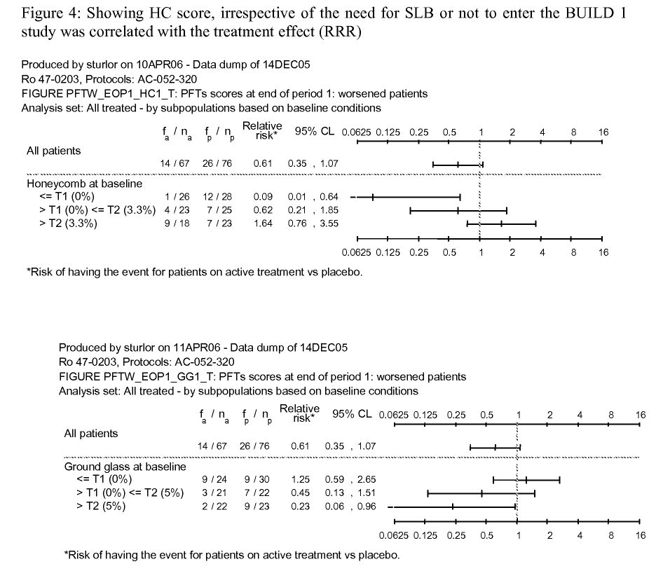

Then we looked at the scoring of honeycombing (HC) vs. the treatment effect. FIG. 4 shows that HC score, irrespective of the need for SLB or not to enter the BUILD 1 study was correlated with the treatment effect (relative risk). The same inverse observation was done for the amount of ground-glass on baseline HRCT. The figure suggests that the maximal treatment effect of bosentan is achieved in patients for whom the HC score is between 0 and 10% of the entire lung fields and/or when ground-glass score is present at patient presentation. The figure also suggests that the maximal treatment effect of bosentan is achieved in patients for whom the HC score is up to 25% of the entire lung fields and/or when ground-glass score is present at patient presentation. This treatment effect may have been obtained also on top of background IPF therapy such as interferon gamma 1b, pirfenidone, imatinib, tumor necrosis factor alpha blocker such as etanercept and N-acetyl cysteine.

In conclusion, the analysis of the BUILD 1 data demonstrates that the dual endothelin receptor antagonist bosentan is mainly effective in the prevention of clinical worsening in IPF patients with early disease with low or no honeycomb on HRCT lung scans.

### REFERENCES

1. Hocher B, Schwarz A, Fagan K A, Thone-Reineke C, El-Hag K, Kusserow H, et al. Pulmonary fibrosis and chronic lung inflammation in ET-1 transgenic mice. Am J Respir Cell Mol Biol 2000;23(1):19-26.
2. Hocher B, Thone-Reineke C, Rohmeiss P, Schmager F, Slowinski T, Burst V, et al. Endothelin-1 transgenic mice develop glomerulosclerosis, interstitial fibrosis, and renal cysts but not hypertension. J Clin Invest 1997;99(6):1380-9.
3. Clozel M, Salloukh H. Role of endothelin in fibrosis and anti-fibrotic potential of bosentan. Annals of Medicine, 2005; 37: 2-12
4. Shi-wen X, Denton C P, Dashwood M R, Holmes A M, Bou-Gharios G, Pearson J D, et al. Fibroblast matrix gene expression and connective tissue remodeling: role of endothelin-1. J Invest Dermatol 2001; 116(3):417-25.
5. Wilson S H, Simari R D, Lerman A. The effect of endothelin-1 on nuclear factor kappa B in macrophages. Biochem Biophys Res Commun 2001;286(5):968-72.
6. Park S H, Saleh D, Giaid A, Michel R P. Increased endothelin-1 in bleomycin-induced pulmonary fibrosis and the effect of an endothelin receptor antagonist. Am J Respir Crit Care Med 1997;156(2 Pt 1):600-8.
7. Mulder P, Richard V, Derumeaux G, Hogie M, Henry J P, Lallemand F, et al. Role of endogenous endothelin in chronic heart failure: effect of long-term treatment with an endothelin antagonist on survival, hemodynamics, and cardiac remodeling. Circulation 1997;96(6): 1976-82.
8. Seccia T M, Belloni A S, Kreutz R, Paul M, Nussdorfer G G, Pessina A C, et al. Cardiac fibrosis occurs early and involves endothelin and AT-I receptors in hypertension due to endogenous angiotensin II. J Am Coll Cardiol 2003;41(4):666-73.
9. Ramires F J A, Sun Y, Mady C, Ramires J A F, Weber K T. Effect of endothelin on myocardial fibrosis in response to chronic administration of angiotensin II or aldosterone. Circulation 1999;100(18):S 2500.
10. Boffa J J, Tharaux P L, Dussaule J C, Chatziantoniou C. Regression of renal vascular fibrosis by endothelin receptor antagonism. Hypertension 2001;37(2 Part 2):490-6.
11. Rockey D C, Chung J J. Endothelin antagonism in experimental hepatic fibrosis. Implications for endothelin in the pathogenesis of wound healing. J Clin Invest 1996;98(6):1381-8.
12. Shi-wen X, Denton C P, Holmes A, Dashwood M R, Abraham D J, Black C M. Endothelins: effect on matrix biosynthesis and proliferation in normal and scleroderma fibroblasts. J Cardiovasc Pharmacol 1998;31(Suppl 1):S360-3.
13. Shi-wen X, et al. Endothelin-1 promotes myofibroblast induction through the ETA receptor via a rac/phosphoinositide 3-kinase/Akt-dependent pathway and is essential for the enhanced contractile phenotype of fibrotic fibroblasts. Mol Biol Cell. 2004 15(6):2707-19.
14. Gomez-Garre D, Ruiz-Ortega M, Ortego M, Largo R, Lopez-Armada M J, Plaza J J, et al. Effects and interactions of endothelin-1 and angiotensin II on matrix protein expression and synthesis and mesangial cell growth. Hypertension 1996;27(4):885-92.
15. Guarda E, Katwa L C, Myers P R, Tyagi S C, Weber K T. Effects of endothelins on collagen turnover in cardiac fibroblasts. Cardiovasc Res 1993;27(12):2130-4.
16. Nakamura T, Ebihara I, Fukui M, Tomino Y and Koide H. Effect of a specific endothelin receptor A antagonist on mRNA levels for extracellular matrix components and growth factors in diabetic glomeruli. Diabetes 1995; 44: 895-899.
17. DaoHH, Lemay J, de Champlain J, deBlois D and Moreau P. Norepinephrine-induced aortic hyperplasia and extracellular matrix deposition are endothelin-dependent. J Hypertension 1973; 19: 1965-1973.
18. Hocher B, George I, Rebstock J, Bauch A, Schwarz A, Neumayer H H, et al. Endothelin system-dependent cardiac remodeling in renovascular hypertension. Hypertension 1999;33(3):816-22.
19. Mulder P, Boujedaini H, Richard V, Derumeaux G, Henry J P, Renet S, et al. Selective endothelin-A versus combined endothelin-A/endothelin-B receptor blockade in rat chronic heart failure. Circulation 2000;102(5):491-3.
20. Mutsaers S E, Foster M L, Chambers R C, Laurent G J, McAnulty R J. Increasedendothelin-1 and its localization during the development of bleomycin-induced pulmonary fibrosis in rats. Am J Respir Cell Mol Biol. 1998;18(5):611-9.
21. Berridge M V, Tan A S. Characterization of the cellular reduction of 3-(4,5-dimethylthiazol-2-yl)-2,5-diphenyltetrazolium bromide (MTT): subcellular localization, substrate dependence, and involvement of mitochondrial electron transport in MTT reduction. Arch Biochem Biophys. 1993;303(2):474-82.
22. Zacharia L C, Jackson E K, Gillespie D G, Dubey R K. Catecholamines block 2-hydroxyestradiol-induced antimitogenesis in mesangial cells. Hypertension 2002; 39 (4): 854-9.

1. A method for the treatment of early stage idiopathic pulmonary fibrosis, wherein honeycomb on HRCT or CT scans is either absent or minimal, comprising administering to a patient in need thereof, bosentan, in free or pharmaceutically acceptable salt form.

2. The method according to claim 1 wherein honeycomb on HRCT or CT scans is present in less than 25% of the overall lung fields.

3. The method according to claim 1 wherein honeycomb on HRCT or CT scans is present in less than 10% of the overall lung fields.

4. The method according to claim 1 wherein the ground-glass attenuation could be any percentage between above zero to 80% of lung fields.

5. The method according to claim 1 wherein bosentan is given to a patient at a daily dosage of 125 mg with or without a lower starting dose.

6. The method according to claim 1 wherein bosentan is given to a patient at a daily dosage of 250 mg with or without a lower starting dose.

7. A method, for the preparation of a medicament for the treatment of early stage idiopathic pulmonary fibrosis, comprising administering to a patient in need thereof, an endothelin receptor antagonist, or a pharmaceutical composition comprising an endothelin receptor antagonist and either pirfenidone or interferon-gamma, in free or pharmaceutically acceptable salt form.

8. The method according to claim 7 wherein the endothelin receptor antagonist is a dual endothelin receptor antagonist or a mixed endothelin receptor antagonist.

9. The method according to claim 7 wherein the endothelin receptor antagonist is a selective endothelin receptor antagonist that binds selectively to the ET_{A} receptor.

10. The method according to claim 7 wherein the endothelin receptor antagonist is a selective endothelin receptor antagonist that binds selectively to the ET_{B} receptor.

11. The method according to claim 7 wherein the endothelin receptor antagonist is selected from table 1.

12. The method according to claim 7 wherein the endothelin receptor antagonist is selected from darusentan, ambrisentan, atrasentan, sitaxsentan, avosentan, TBC-3711, tezosentan, clazosentan, propyl-sulfamic acid {5-(4-bromo-phenyl)-6-[2-(5-bromo-pyrimidin-2-yloxy)-ethoxy]-pyrimidine-4-yl}-amide and bosentan.

13. The method according to claim 7 wherein the endothelin receptor antagonist is selected from darusentan, ambrisentan, sitaxsentan, avosentan, TBC-3711, propyl-sulfamic acid {5-(4-bromo-phenyl)-6-[2-(5-bromo-pyrimidin-2-yloxy)-ethoxy]-pyrimidine-4-yl}-amide and bosentan.

14. The method according to claim 7 wherein the endothelin receptor antagonist is bosentan.

15. The method according to claim 7 wherein honeycomb on HRCT or CT scans is either absent or minimal.

16. The method according to claim 7 wherein honeycomb on HRCT or CT scans is present in less than 25% of the overall lung fields.

17. The method according to claim 7 wherein honeycomb on HRCT or CT scans is present in less than 10% of the overall lung fields.

18. The method according to claim 7 wherein the ground-glass attenuation could be any percentage between above zero to 80% of lung fields.

19. The method according to claim 14 wherein bosentan is given to a patient at a daily dosage of 125 mg with or without a lower starting dose.

20. The method according to claim 14 wherein bosentan is given to a patient at a daily dosage of 250 mg with or without a lower starting dose.
